(19) United States
(12) Patent Application Publication
(10) Pub. No.: US 2024/0131487 A1
(43) Pub. Date: Apr. 25, 2024

(54) GAS-LIQUID-SOLID AND LIQUID-SOLID REACTOR CASCADE FOR CARRYING OUT CONTINUOUS-FLOW CHEMICAL REACTIONS UNDER HIGH PRESSURE AND/OR HIGH TEMPERATURE

(71) Applicant: IPSOMEDIC, MARSEILLE (FR)

(72) Inventor: Edith LECOMTE-NORRANT

(21) Appl. No.: 18/554,911

(22) PCT Filed: Apr. 27, 2022
(86) PCT No.: PCT/EP2022/061243

§ 371 Date: Oct. 11, 2023

(30) Foreign Application Priority Data
Apr. 27, 2021 (FR) 2104386

(51) Int. Cl. B01J 3/04; B01J 19/00; B01J 19/18; B01J 21/06; B01J 21/18; B01J 23/42; B01J 23/44; B01J 25/02; B01J 29/70; C07C 1/32; C07C 45/29; C07C 45/45; C07C 51/347; C07C 209/48; C07C 213/02; C07D 207/27; C07D 317/36; C07D 333/08

(52) U.S. Cl. CPC B01J 3/04; B01J 19/0013; B01J 19/0066; B01J 19/1862; B01J 21/06; B01J 21/18; B01J 23/42; B01J 23/44; B01J 25/02; B01J 29/7007; C07C 1/321; C07C 45/29; C07C 45/455; C07C 51/347; C07C 209/48; C07C 213/02; C07D 207/27; C07D 317/36; C07D 333/08; B01J 2219/00094; B01J 2219/00186

(57) ABSTRACT

The present invention relates to a device for carrying out continuous-flow chemical reactions under pressure or high pressure using a cascade of perfectly stirred Gas-Liquid-Solid reactors, and to the use of these devices for the implementation of such reactions. The device comprises a cascade of interconnected autoclave reactors. The reactors of the cascade are of different volumes and are provided with means allowing them to be controlled individually in a completely independent manner. The cascade of reactors comprises at least two reactors of different volumes, increasing or decreasing in the fluid flow direction.

### TECHNICAL FIELD

The present invention relates to a device making it possible to carry out chemical reactions under pressure or high pressure in continuous flow by a cascade of perfectly stirred Gas-Liquid-Solid reactors and the use of these devices for the implementation of such reactions.

### STATE OF THE ART

Chemical reactions under high pressure (hydrogenations, oxidations, carbonylations, etc.) represent a large part of the transformations implemented on an industrial scale, in the field of both basic chemicals (petrochemicals) and fine chemicals (industrial pharmaceutical, cosmetic, etc.). On their own, hydrogenation reactions using dihydrogen under pressure represent approximately 20% of the chemical reactions carried out in the world of fine chemicals (A. van den Berg et al. Tetrahedron 2005, 61, 2733-2742).

These same chemical transformations carried out under very high pressure (from a few tens to several hundreds of bars) are extremely dangerous due to the very large volumes of the reactors used (up to several thousand litres) to meet market needs. The highly flammable nature of the gases used (hydrogen, oxygen) or highly toxic (ammonia, carbon monoxide) require compliance with a large number of restrictive regulatory measures prior to the establishment of an industrial unit for chemical reactions under high pressure. (high threshold in Seveso classification).

It is now commonly accepted that the transition from a conventional batch process (sequences loading the reactor - reaction - emptying - cleaning) to a continuous flow process (feeding and withdrawal from the reactor without interruption) allows a considerable reduction in risks and a better control of the dangerousness of the risk, in particular due to the considerable reduction in the involved volumes (I. R. Baxendale et al. J. Chem. Technol. Biotechnol. 2013, 88, 519-552). Intrinsic safety is thus obtained.

The drastic reduction in the surface to volume ratio, a consequence of the continuous passage, makes it possible to significantly improve gas-liquid, liquid-solid and gas-solid transfers (material and heat) which are always key parameters to be mastered to designate a competitive pressure reaction industrial unit (J.-C. M. Monbaliu et al. Eur. J. Org. Chem. 2018, 2301-2351). This characteristic generally translates into a considerable improvement in the performance of a continuous device compared to the equivalent batch process (yield, selectivity, productivity, environmental discharge, energy balance).

The possibility of integrating into a continuous production unit, an online analysis system in order to visualize in real time the level of performance of the device makes it possible to correct any malfunction almost instantaneously, which leads to a considerable improvement in safety. and quality.

However, the implementation of a chemical reaction under high pressure presents a major difficulty in that this type of transformation is almost systematically carried out in a three-phase medium (gas, liquid, solid catalyst) and that it is generally impossible to convey a solid phase in a piston-type continuous reactor without altering it (C. O. Kappe et al. Chem Sus Chem 2011, 4, 300-316).

To overcome this problem, several innovative devices have been designed in the academic environment as in the industrial environment allowing the implementation of chemical reactions under high pressure in continuous flow.

The use of macroporous or mesoporous monolithic reactors makes it possible to directly convey the substrate to be transformed through the pores of the materials supporting the solid catalyst (Chem. Eng. Sci. 2001, 56, 6015-6023).

The use of reactors pre-packaged in catalytic cartridges, tubular reactors coated with catalyst or fixed catalytic beds also constitute a solution of choice for the implementation of these reactions (Duprat F. et al., Org. Proc. Res. Dev. 2020, 24, 686-694 ; J. Comb. Chem. 2008, 10, 88-93; U.S. Pat. No. 7,988,919; International application WO 2017106916).

The use of so-called “slurry” reactors makes it possible to achieve a three-phase continuous flow ensuring good contacting of the reactants with the active sites of the catalyst (U.S. Pat. No. 8,534,909, Chemical Engineering Journal 2011, 167 (2-3), 718- 726., International Application W02007/112945).

However, these devices are often not very flexible and suffer from major practical drawbacks such as problems of clogging, fouling, “leaching” or even technical maintenance difficulties (changing the catalyst). Even more limiting is the poor compatibility of these devices with industrial catalysts, which are generally used in conventional batch reactors, both in terms of particle size (presence of small and large solid particles between 5 μm and 350 μm) of crystalline nature or physico-chemical properties.

### DISCLOSURE OF THE INVENTION

The inventors have designed a new device allowing chemical reactions to be carried out under pressure or high pressure and/or under high temperature in continuous flow on the basis of a cascade of N (natural whole N greater than 1) autoclave reactors gas-liquid-solid or liquid-solid perfectly agitated and interconnected.

The device is perfectly flexible and can tolerate a reactive gas pressure of 10 to 500 bars, a temperature of −30 to 300° C. and is compatible with all types of heterogeneous catalyst (particle size from 2 μm to 500 μm) with catalytic loads that can be significant (from 0.1% to 5% w/w or even 10%) as well as a wide range of residence times, from a few minutes to several hours.

The Gas-Liquid-Solid (GLS) and Liquid-Solid (LS) devices of the invention make it possible to work under optimized conditions according to the kinetics of the reaction.

As will be shown in the experimental part, the devices of the invention demonstrate great flexibility, unlike existing systems which operate continuously on the solid and liquid phases, therefore with constant concentrations of catalysts and the settings of which cannot be varied.

The Gas-Liquid-Solid (GLS) devices of the invention make it possible to vary the catalyst load in line with the kinetics of the reaction and of the gas-liquid transfer, which provides a great flexibility to these devices.

The subject of the invention is therefore a device for chemical reactions under pressure or high pressure in continuous flow comprising a cascade of N autoclave reactors interconnected, characterized in that the N reactors of the cascade have different volumes and are provided with means for controlling them individually in a completely independent manner, it being understood that N is a natural integer greater than 1 and that the cascade of reactors preferably comprises at least two reactors of different volumes, increasing or decreasing in the direction of the flow of the fluids.

It is understood that the invention also relates to devices comprising a cascade of reactors of different volumes in which the reactions are carried out under different conditions depending on the reactors in terms of volume of reaction medium, temperature, pressure of reactive gas, catalyst concentration and/or stirrer rotational speed.

The present invention relates to a device for chemical reactions under pressure or high pressure and/or under high temperature in continuous flow comprising a cascade of N autoclave reactors interconnected, characterized in that the N reactors of the cascade are provided with means allowing them to be individually controlled in a completely independent manner, it being understood that N is a natural integer greater than 1 and that the cascade of reactors comprises at least two reactors of different volumes, increasing or decreasing in the direction of the flow of the fluids, the said chemical reactions being of the Gas-Liquid-Solid type or of the Liquid-Solid type, said device comprising between each of said reactors means allowing the fluid phase to be in continuous flow and allowing the solid phase to be in batch.

By “under pressure” is meant pressures greater than several hundred thousand pascals, which correspond to usual pressures in the context of chemical reactions that cannot be carried out in borosilicate glass reactors used by those skilled in the art, because they do not resist these pressures.

By “high pressure” is meant pressures greater than 1 MPa, and which corresponds to pressures encountered when one of the reactants is a gas.

The expression “high temperature” corresponds to temperatures above approximately 50° C.

The expression “in continuous flow” means the implementation of a chemical reaction in reactors through which a flowing liquid reaction medium passes, and in which all the stages of this specific chemical reaction are carried out without isolating the intermediates in order to obtain a complete conversion of one of the reactants and/or to obtain the desired product. By “cascade of reactors”, is meant a succession of several reactors in a certain consecutive order, each reactor being dedicated to the conversion of one or more stages of a determined chemical reaction, and all of these stages in the consecutive reactors in this order allowing said chemical reaction to be carried out.

The expression “autoclave reactor” designates a reactor capable of withstanding a pressure of several hundred thousand pascals while being continuous on the liquid and gas phases.

N represents the number of reactors and is a natural integer greater than or equal to 2, advantageously from 2 to 10, and which can take the values 2, 3, 4, 5, 6, 7, 8, 9 or 10.

A continuous flow assembly requires at least 2 reactors to be qualified as such.

The number of reactors in the cascade cannot exceed 10.

Indeed, each additional reactor involving a pressure drop compared to the previous reactor, in particular a loss of approximately 0.3 to 2 bars (0.03 to 0.2 MPa) per reactor, this would lead to a significant reduction in the speed of reaction in the last reactors on a cascade of more than 10 reactors.

The expression “making it possible to individually control each reactor” means controlling the pressure, the temperature, the volume of the liquid, and above all the composition of the reaction medium. In fact, monitoring the composition of the reaction medium as a function of time makes it possible to monitor the kinetics of the reaction and to control the activity of the catalyst as a function of time and to plan to change the catalyst load when the catalyst is sufficiently deactivated and no longer meets the required quality criteria, that is to say the conversion rate of the expected reaction.

The unloading of the spent catalyst load from reactor n takes place quickly, approximately 15 minutes to 1 hour via the bottom valve depending on the reaction volume of reactor n.

Indeed, before unloading the catalyst through the bottom valve, the reactor must be inerted with an inert gas (Nitrogen, Argon, etc.) then the reactor must be completely drained of the liquid and solid phases and cleaned before recharging the reactor with a new catalyst and the reintroduction of the reaction medium from reactor n−1.

This reactor n is bypassed during this step of unloading the deactivated catalyst, cleaning and loading the new catalyst while the other reactors are in operation.

The expression “totally independent” means that parameters such as the pressure or the temperature of each reactor do not influence the operation of the other reactors.

The expression “different volumes” means that a reactor has a volume difference of at least 5% compared to another reactor of this cascade.

In other words, if the difference in volume between two reactors is less than 5%, the reactors are considered to have the same volume.

By “increasing or decreasing volumes”, is meant the fact that the volume of the reactors of the cascade can be strictly increasing or decreasing according to the direction of the cascade.

It is also means that several reactors of this cascade can be of the same volume, provided that in the cascade there is at least one reactor of volume less or greater than the volume of the reactors of the same volume.

One also means that one can have a series of reactors of increasing or identical volumes, followed by one or more reactors of decreasing volumes.

Finally, one means that one can have a series of reactors of decreasing or identical volumes, followed by one or more reactors of increasing volumes.

“Direction of fluid flow” means that the fluid flow circulates in a single direction, traversing the entire cascade of reactors from the first reactor to the last reactor in a direction defined by the user.

“First reactor” means the reactor into which the fresh raw materials are inserted.

The expression “Gas-Liquid-Solid reaction” means that one or more reactants are in a gas form, one or more reactants are in a liquid form and at least one of the reactants, or a catalyst is in a solid form.

By “Liquid-Solid reaction”, it is meant that one or more reactants are in a liquid form and at least one of the reactants, or a catalyst is in a solid form.

The expression “in batch” means that the catalyst or the solid reagent remains in the reactor into which it is introduced during the reaction

A particular subject of the invention is a device characterized in that each reactor is provided with a liquid inlet and outlet, and with a possible reactive gas inlet, a bursting disc, a vent, an immersion sleeve for measuring parameters, a sampling valve, a double jacket, a heating collar and a valve placed at the bottom of each reactor and making it possible to draw off the deactivated catalyst and to replace it with a new catalyst.

The invention particularly relates to a device as defined above, characterized in that each reactor is provided with a liquid inlet and outlet, and with a possible reactive gas inlet, a bursting disc, a vent, an immersion sleeve for parameter measurement, a sampling valve, a double jacket, a heating collar and a valve placed at the bottom of each reactor and making it possible to withdraw the deactivated catalyst and to replace it with a new catalyst, each reactor being equipped with a filter, in particular a frit inside the reactor on the liquid outlet in order to ensure the separation of the solid-liquid and maintaining the solid in the reactor, so that the solid phase is in batch and the liquid phase is continuous.

Each reactor is generally and preferably equipped with counter blades.

The liquid coming from the liquid outlet of each reactor is also called clear liquid because thanks to the filter system with which each reactor is equipped, there is no longer any trace of solid.

The term “filter” refers to a wall with pores allowing fluids to pass but retaining solids.

This allows solid catalysts and reactants to remain within the reactor and not circulate with the flow of fluids.

In particular, this allows the catalyst to be used completely until it is deactivated.

A particular object of the invention is a device characterized in that each reactor is equipped with a reactive gas inlet, a second gas inlet between each reactor to remove the catalyst from the frit, an inlet and outlet for the liquid, a bursting disc, a vent, an immersion sleeve for parameter measurement, a sampling valve, a jacket, a heating collar and a valve placed at the bottom of each reactor and making it possible to withdraw the deactivated catalyst and to replace it with a new catalyst.

The pressure drop of around 0.3 to 2 bars between the 2 reactors in series, mainly due to clogging of the filter at the liquid phase outlet, can be compensated by adding an inert gas (Argon, Nitrogen . . . ) in order to maintain the reactor n under the pressure necessary for the reaction and to carry out in said reactor n, the transfer of the liquid phase continuously from the reactor n to the reactor n+1.

The subject of the invention is in particular a device as defined above, characterized in that the liquid outlet orifice is fitted with a system of filter candles, with a porosity of between 2 and 50 μm.

The expression “filter candle” means a filter, hollow and porous cylinder with a large exchange surface and a porosity adapted to the solid phase, that is to say having a porosity less than the size of the solid crystals to retain this solid phase in the reactor and obtain a clear liquid at the outlet of the reactor.

The subject of the invention is in particular a device as defined above, characterized in that an online analysis tool PAT (Process Analytical Technology) by UV, NIR, Raman or any other analysis technique is positioned between each reactor.

The device can be equipped with an online analysis system (UV, RAMAN or NIR probe or any other analysis technique) to visualize in real time the proper functioning of the process in progress.

“online analysis tool PAT” means a set of on-line spectroscopic and chromatographic composition analyzers, fixed-use sensors and automated and statistical data analysis in order to control the continuous process in order to obtain the quality of the finished product, without the need to take samples.

In a particular embodiment of the device, the outlet orifice is provided with a system of filter candles, with a porosity of between 2 and 50 1.tm and an online analysis tool PAT (Process Analytical Technology) by UV, NIR or Raman, which is positioned between each reactor.

Such a device allows the implementation of a continuous process on the liquid phase (supply of substrate and withdrawal of product) and batch on the solid phase.

Indeed, a system of filter candles positioned between each reactor of the cascade makes it possible to keep constant the catalytic load specific to each reactor.

The particular object of the invention is a device characterized in that the implementation of the method is in continuous flow with regard to the liquid phase and in batch with regard to the solid phase.

The device is provided with a high level of control in that the individual parameters (temperature, pressure, stirring, catalyst load) of each reactor of the cascade can be independently controlled.

An efficient liquid gas transfer is ensured in each reactor of the cascade by a system of self-priming turbine and counter blades.

The device can be used for carrying out any type of chemical reaction under pressure or high pressure, mainly hydrogenation reactions but also oxidation, carbonylation or even amination reactions.

The device can be used in continuous mode by connecting 1 to N (N natural integer) reactors in cascade or in batch mode by using a single closed reactor and to which it refers in the context of the present invention to present comparative results.

The device as defined above for carrying out reactions under pressure or high pressure can be characterized in that in the cascade of reactors, the volume of the reactors is decreasing and is such that when N is equal to or greater than 3, if the first reactor has a volume R1, the second reactor has a volume R2 comprised between R1 and 0.5 R1 and the third reactor has a volume R3 comprised between 0.8 R1 and 0.4 R1.

It is for example possible to operate with a cascade of reactors of decreasing volumes in the following proportions : 1, 0.75, 0.5.

This type of device in which the cascade of reactors has a decreasing volume in the direction of fluid flow is preferably used for the implementation of reactions the reaction heat of which is less than 50 kJ/mol such as, in general, reactions of saponifications or of retro-esterifications, the reaction rates of which can be accelerated by increasing the temperature.

The present invention also relates to a device for carrying out reactions under pressure or high pressure characterized in that in the cascade of reactors, the volume of the reactors is increasing and is such that when N is equal to or greater than 3, if the the first reactor has a volume R1, the second reactor has a volume R2 comprised between 1.25 R1 and 1.5 R1 and the third reactor has a volume R3 comprised between 1.5 R1 and 4 R1.

It is for example possible to operate with a cascade of reactors of increasing volumes in the following proportions: 1; 1.5 and 4.

This type of device in which the cascade of reactors has an increasing volume in the direction of fluid flow is preferably used for the implementation of reactions, the reaction heat of which is greater than 50 kJ/mol such as catalytic hydrogenations or oxidations.

As a general rule, a cascade of reactors of increasing volumes is used when the reaction heat is high, for example greater than 50 kJ/mol and/or when the reaction kinetics become very slow when the conversion is greater than 40%.

It is then necessary to increase the residence time to obtain an optimal volumetric productivity with the possibility of increasing the catalyst load and the temperature.

A particular object of the invention is a use as defined above for carrying out reactions under pressure or high pressure of the liquid-solid-gas and solid-liquid type, in particular hydrogenation, oxidation, carbonylation, carboxylation, amination, in particular ammonolysis, Heck or Suzuki-Miyaura, preferably hydrogenation reactions.

A particular object of the invention is a use as defined above for carrying out reactions under pressure or high pressure of the liquid-solid-gas type, in particular hydrogenation, oxidation, carbonylation, carboxylation or amination, especially ammonolysis, preferably hydrogenation reactions.

A particular object of the invention is a use as defined above for carrying out reactions at high temperature of the solid liquid type, in particular Heck and Suzuki-Miyaura reactions.

A particular object of the invention is a device characterized in that each reactor is provided with stirring by a hollow self-priming turbine ensuring dispersion of the reactive gas in the reaction medium thanks to a depression created by the blades of the stirrer and in that the stirring speed is preferably greater than 300 rpm.

A particular object of the invention is a use as defined above, characterized for carrying out Gas-Liquid-Solid reactions, in which each reactor is provided with stirring by a hollow self-priming turbine ensuring dispersion of the reactive gas in the reaction medium thanks to a depression created by the blades of the agitator and in that the stirring speed is sufficient to overcome the pressure drop and is preferably greater than 300 rpm, in particular 500 rpm.

The expression “self-priming turbine” designates a turbine with a hollow axis of rotation which sucks up the reactive gas present in the gas phase of the reactor to disperse it in the liquid phase at the bottom of the reactor, behind the stirring blades.

This phenomenon is induced by the depression behind the stirring blades when the rotation speed is greater than 300 rpm or even greater than 500 rpm to combat the pressure drop due to the height of the liquid in the reactor.

When the stirring speed is not sufficient to overcome the pressure drop due to the height of the liquid, there is no recirculation of the gas phase in the upper part of the reactor in the liquid phase located at the bottom of the reactor, at the level of the blades of the agitator, and the gas-liquid transfer is strongly reduced, which can induce a strong reduction in the rate of reaction.

By “stirring” it is understood that the liquid phase within the reactor is mixed in such a way as to make it as homogeneous as possible, in particular to make the reaction medium as homogeneous as possible in temperature, in concentration with a suspension of the catalyst as dispersed and homogeneous. Indeed the presence of immiscible liquids can create two phases within the reactor. This also makes it possible to suspend a solid in a liquid phase, when one of the reactants is a solid and/or when a heterogeneous catalyst is required.

In the case of a Gas-Liquid-Solid reaction, this can also allow the gas to be dispersed within the liquid.

The invention also relates to a device characterized in that an N+1th reactor can be positioned at the end of the cascade and connected to the process during maintenance operations requiring one of the reactors of the cascade to be isolated.

A particular object of the invention is a device as defined above, characterized in that an N+1th reactor can be positioned at the end of the cascade and connected to the process during occasional maintenance operations requiring one of the reactors of the cascade to be isolated.

The expression “one-off maintenance operation” means changing the catalyst in one of the reactors or repairing a failure in the temperature, pressure or PAT sensor control system.

The invention relates to the use as defined above characterized in that the reaction is a Gas-Liquid-Solid reaction, implemented so that the reactive gas pressure is between 2 bars (0.2 MPa) and 500 bars (50 MPa) preferably between 2 bars (0.2 MPa) and 250 bars (25 MPa) and more preferably between 2 (0.2 MPa) and 50 bars (5 MPa).

The invention relates to the use in a Gas-Liquid-Solid reaction as defined above characterized in that the reaction temperature is between −10 and 300° C., preferably a high temperature of at least 130° C., preferably by using either a double jacket or a heating collar, and in that the reaction temperature and the catalyst load can be different in each of the reactors.

The invention relates to the use as defined above, characterized in that the reaction is a Liquid-Solid reaction, characterized in that the reaction is implemented so the reactive gas pressure is between 1 bar (0.1 MPa) and 100 bars (10 MPa) preferably between 1 bar (0.1 MPa) and 50 bars (5 MPa) and more preferably between 1 bar (0.1 MPa) and 30 bars (3 MPa).

The invention relates to the use in a Liquid-Solid reaction as defined above, characterized in that the reaction temperature is between −10 and 300° C., preferably a high temperature of at least 130° C. preferably by using either a double jacket or a heating collar, and in that the reaction temperature and the catalyst load can be different in each of the reactors.

The invention relates to the use as defined above of the continuous hydrogenation reaction of adiponitrile to hexamethylene diamine in the presence of Raney nickel, characterized in that the process is implemented by using at least three reactors of different volumes with decreasing volumes and increasing masses of catalysts and temperatures depending on the reactors.

The invention relates to the use of a device as defined above, characterized in that the cascade of reactors comprises three elements and in that the volume of the reactors is decreasing and is such that if the first reactor has a volume R1, the second reactor has a volume R2 equal to half of R1 and the third reactor has a volume R3 equal to a third of R1.

The invention relates to the use as defined above for the continuous hydrogenation reaction of p-nitrophenol to p-aminophenol in the presence of a platinum-on-carbon catalyst (Pt/C) characterized in that the process is implemented by using a cascade of two to five reactors, preferably with a decreasing hydrogen pressure depending on the reactors.

The invention relates to the use as defined above for the continuous acetylation reaction of anisole to acetanisole using acetic anhydride in the presence of beta zeolite, characterized in that the process is implemented by using a cascade of at least two reactors and at a temperature of at least 130° C.

The invention relates to the use as defined above for the continuous ammonolysis reaction of ethyl 2-(2-pyrrolidone)-butyrate to 2-(2-oxopyrolidin-1-yl)butyramide in the presence of sodium methanolate characterized in that the process is implemented by using a cascade of at least two reactors at a pressure of at least 7.5 bars (0.75 MPa) and at a temperature of at least 117° C.

The invention relates to the use as defined above for the continuous oxidation reaction of benzyl alcohol to benzaldehyde using a SiliaCat Pd(0) palladium catalyst, characterized in that the process is implemented by using a cascade of at least 2 reactors and at a pressure of at least 10 bars (1 MPa), in particular at a temperature of 85° C.

The invention relates to the use as defined above for the carboxylation reaction of propylene oxide to propylene carbonate using a diethylaminoethyl cellulose catalyst, characterized in that the process is implemented by using a cascade of at least two reactors and at a pressure of at least 7 bars (0.7 MPa) and at a temperature of at least 95° C.

The invention relates to the use as defined above for the continuous Suzuki-Miyaura reaction of a boronic acid with an iodoaryl using a Pd—Cu/C catalyst, characterized in that the process is implemented by using a cascade of at least two reactors and at a temperature of at least 105° C., in particular at a pressure of 2 bars (0.2 MPa).

The invention relates to the use as defined above for the continuous Heck reaction of an alkenyl or an alkyne with an iodoaryl using a palladium Pd-M/C catalyst with M a metal, characterized in that the process is implemented by using a cascade of at least two reactors and at a temperature of at least 105° C., in particular at a pressure of 4 bar (0.4 MPa).

### BRIEF DESCRIPTION OF THE DIAGRAMS

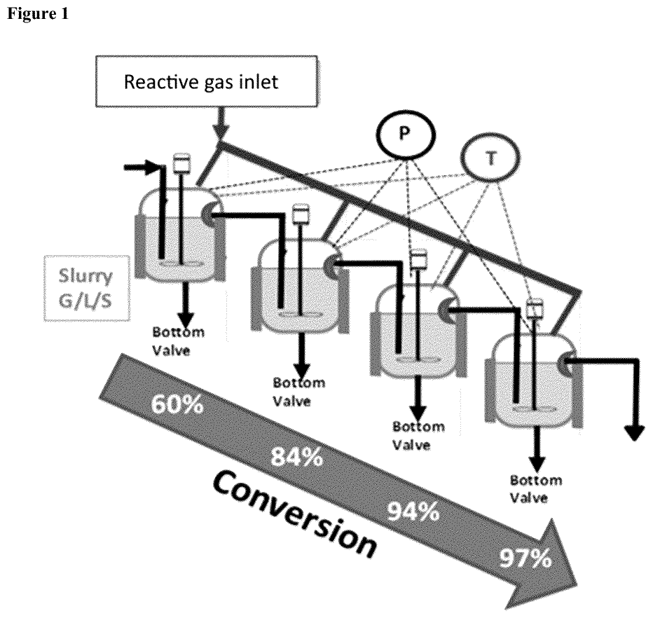

FIG. 1 shows a simplified block diagram of the device in the context of a reaction under pressure where the residence time would be set so as to have a conversion of 60% in the first reactor of a cascade of 4 reactors.

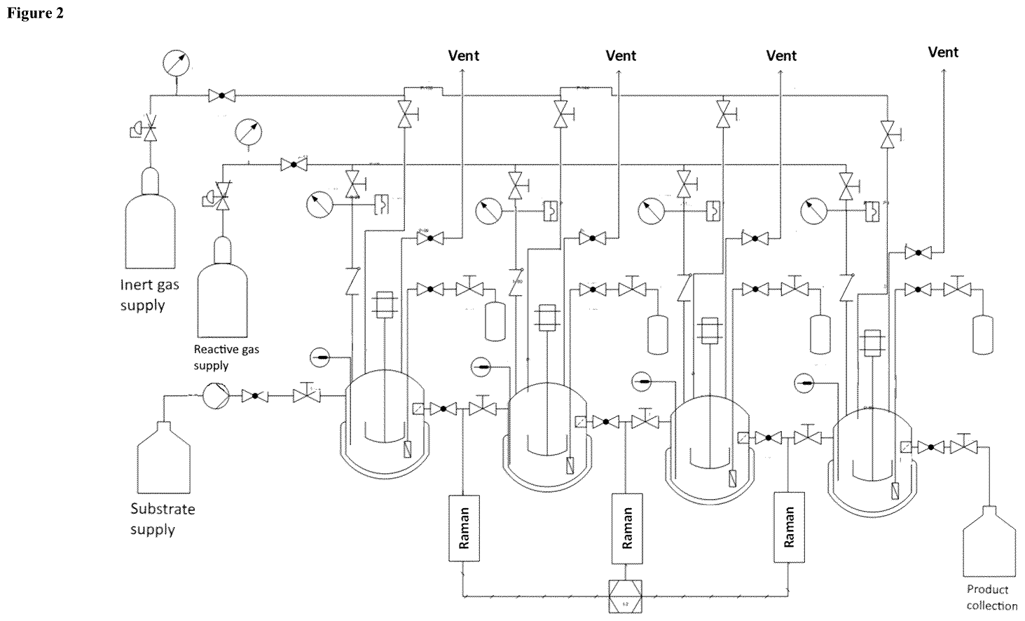

FIG. 2 represents a PI&D diagram (piping and instrumentation diagram) of the complete device used for a continuous process in the case where N=4 (cascade of 4 reactors).

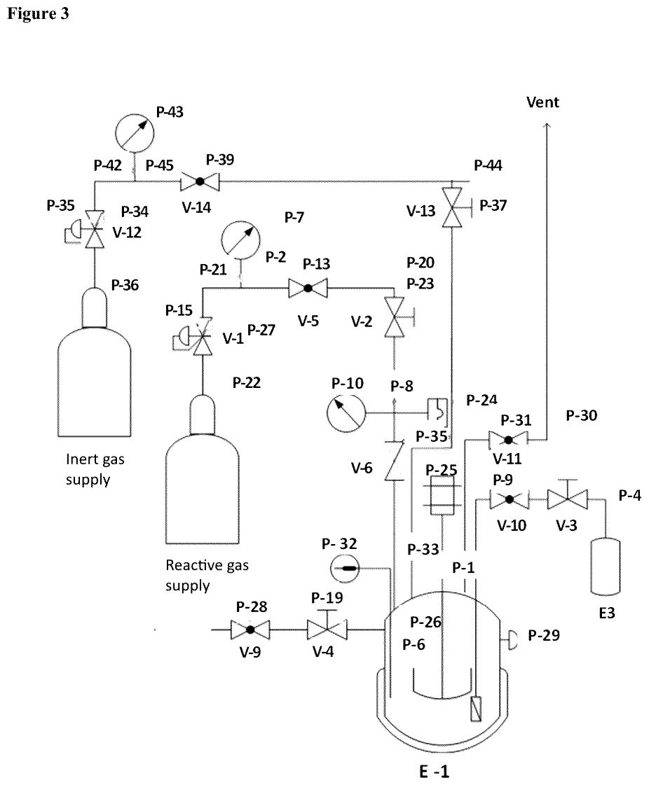

FIG. 3 represents a PI&D diagram of the complete device used for a batch process on a single closed reactor.

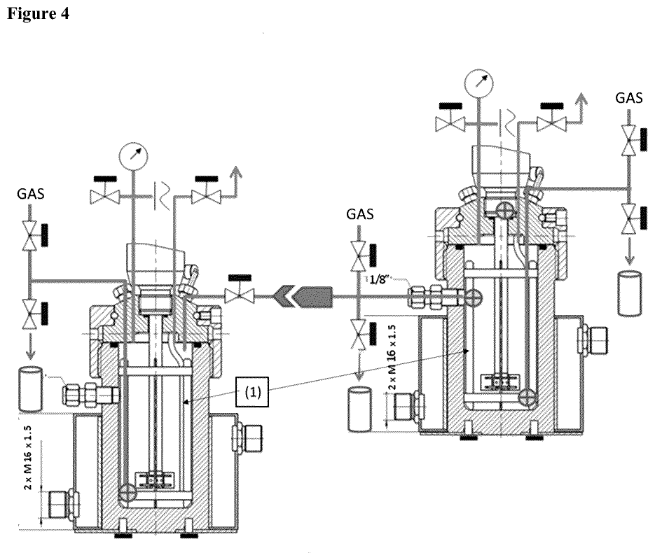

FIG. 4 represents a sectional view of the complete device used for a continuous process in the case where N=2 (cascade of 2 reactors). (1) represents the counter blades.

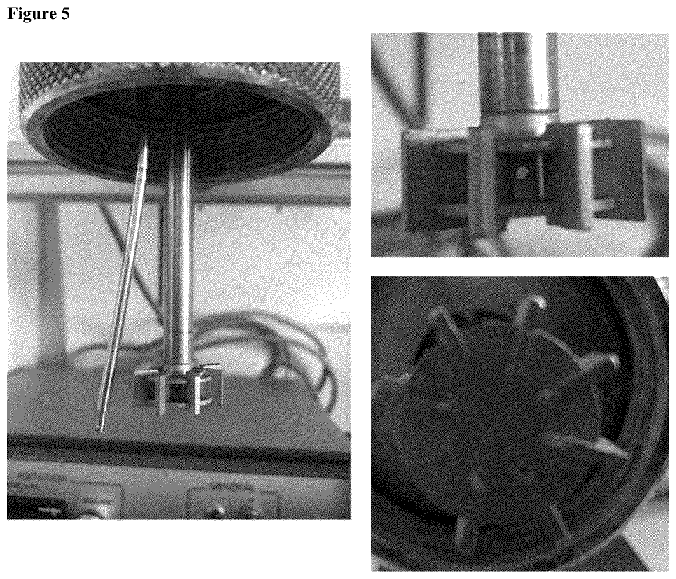

FIG. 5 represents photographs of the self-priming Rushton turbine

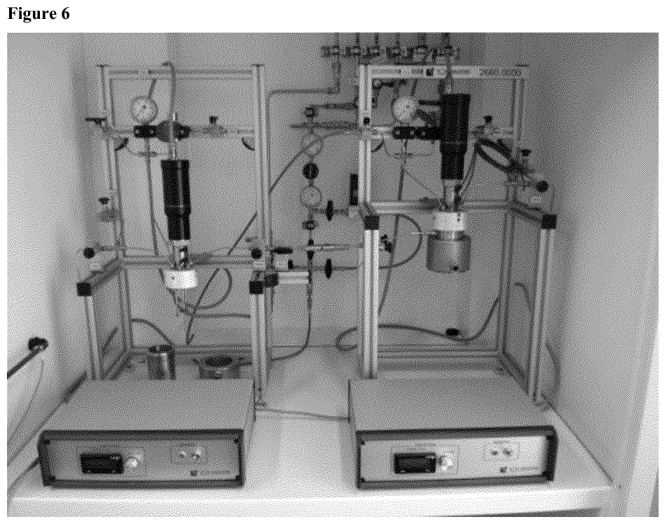

FIG. 6 represents a photograph of the complete device used for a continuous process in the case where N=2 (cascade of 2 reactors).

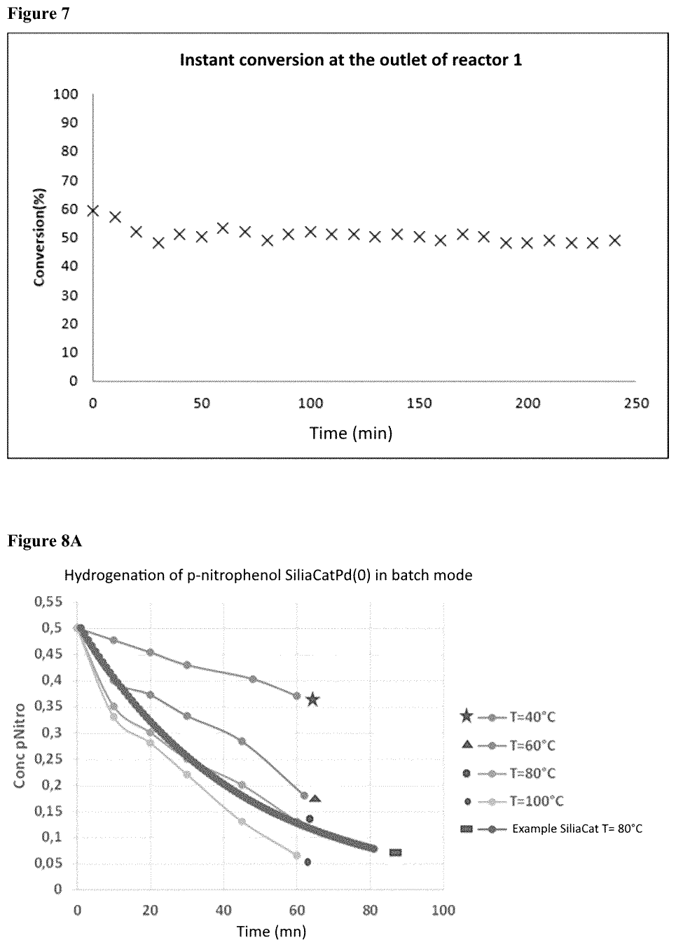

FIG. 7 represents a graph of the conversion (measured by HPLC) of the hydrogenation reaction of p-nitrophenol to p-aminophenol carried out continuously under the conditions set out in Example 2.

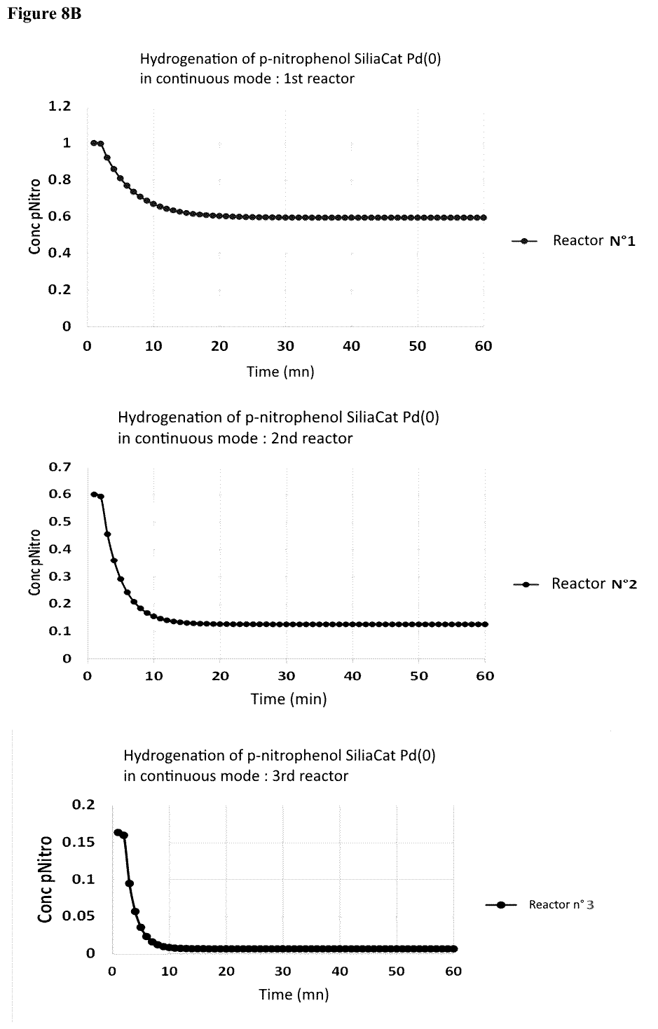

FIG. 8A represents a graph of the conversion (measured by HPLC) of the hydrogenation reaction of p-nitrophenol to p-aminophenol carried out in batch and FIG. 8B continuously under the optimized conditions in the 3 reactors in series.

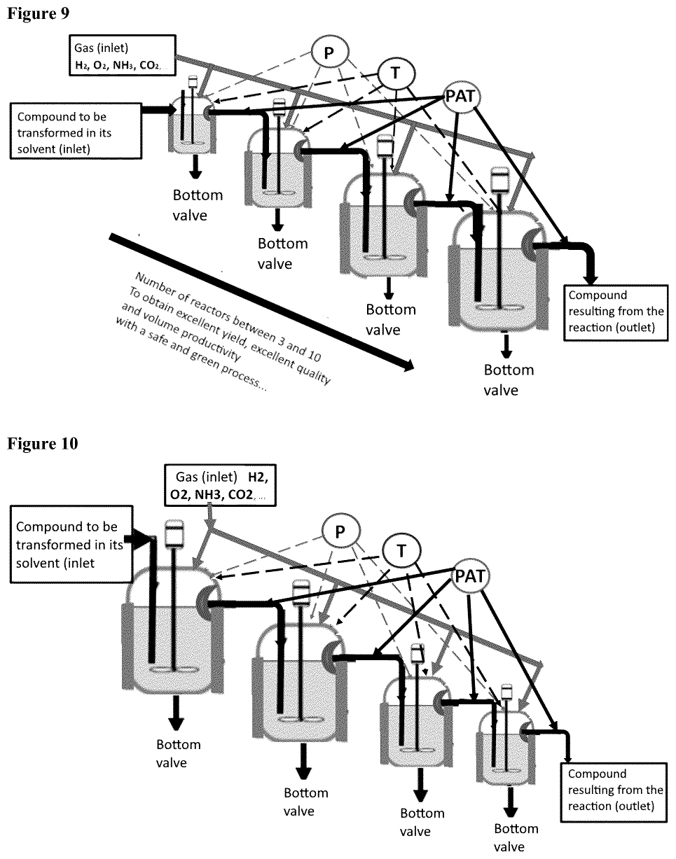

FIG. 9 represents a PI&D diagram (piping and instrumentation diagram) of the complete device used for a continuous process in the case where N=4 (cascade of 4 reactors of increasing size).

FIG. 10 represents a PI&D diagram (piping and instrumentation diagram) of the complete device used for a continuous process in the case where N=4 (cascade of 4 reactors of decreasing size).

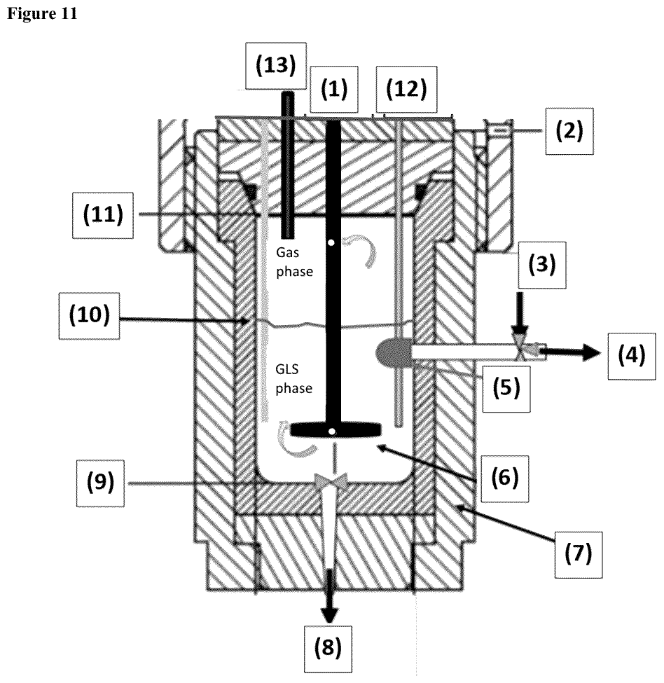

FIG. 11 represents a diagram of a reactor of the cascade of reactors used for a Gas-Liquid-Solid reaction. (1) represents the axis of stirring. (2) represents the continuous gas input to maintain a constant pressure in the reactor. (3) represents the point gas entry when the frit is covered with catalyst and induces a pressure drop between the 2 reactors in cascade.

This makes it possible to resuspend the catalyst in the reactor and to maintain a constant liquid volume in the reactor (4) represents the clear liquid at the outlet of the reactor to enter reactor n+1 continuously. (5) represents the frit. (6) represents the self-priming turbine. (7) represents the double jacket for temperature control. (8) represents the deactivated catalyst outlet. (9) represents the valve to evacuate the catalyst when it is deactivated. (10) represents the liquid level. (11) represents the continuous liquid inlet. (12) represents the liquid sample intake (13) the inlet for installing the sensors (Temperature, Pressure, PAT Control).

### DETAILED DESCRIPTION OF THE INVENTION

The device consists of a cascade of autoclave reactors Gas-Liquid-Solid perfectly agitated individually identical and connected to each other by fluidic connections equipped with filter candles (FIG. 1).

Each reactor consists of a cylindrical stainless steel tank, the volumes of which are between 100 ml and 200 liters.

A preferred value for the volume of the reactors at the laboratory/pilot level is 250 ml (FIG. 6) and at the industrial level is between 2 liters and 200 liters.

According to an embodiment, the dimensions of each reactor range from 45 mm to 80 cm in internal diameter by 9 cm to 100 cm in height for a total volume of 150 mL to 200 L. Preferably, the dimensions are from 45 to 500 mm in internal diameter by 95 to 600 mm in height for a total volume of 150 mL to 120 L. Advantageously, the external diameter of each reactor can be greater in the case of reactions under a particularly high pressure.

According to an embodiment, the tightness of each reactor is ensured by an O-ring of the VITON type or equivalent compatible with the products used and the temperature.

The reactor is closed by an obturator equipped with screw nuts, adapted to the volume of the reactor and the operating conditions to maintain the pressure in the reactor.

According to an embodiment, sealing is ensured by a system of gaskets compressed by a system of flanges.

This operating mode is preferred in the case of very high pressure processes (greater than 200 bars (20 MPa)).

The shutter of each reactor is traversed by a motorized drive shaft connected to an individual control box making it possible to adjust the stirring speed between 0 rpm and 1200 rpm.

Advantageously, the shutter of each reactor as well as the motorized stirring turbine are fixed on a frame, preferably in stainless steel adapted to the size of the reactors (FIG. 6)

According to an embodiment, the shutter of each reactor is equipped with 4 to 8 nozzles ⅛″ HP (0.3175 cm) or even ¼″ (0.635 cm), or 1″ (2.54 cm) in depending on the needs, preferably 4 to 6 nozzles (FIG. 3).

One of the shutter nozzles is connected by ⅛″ (0.3175 cm), ¼″ (0.635 cm), or 1″ (2.54 cm) stainless steel tubing to a junction in 4-way cross of the Swagelok type, marketed by the Swagelok Company (FIG. 3).

The diameters of the tubes are in line with the reaction volumes and therefore will be greater when the reaction volumes are of the order of 10 liters to 150 liters, preferably from 10 to 50 liters.

One of the paths of this cross connection is connected to the reactive gas supply.

Another of the channels of this cross connector is connected to an electronic (and/or needle) pressure gauge to measure the reactive gas pressure in the reactor and allows the recording of the pressure as a function of time.

The third port of this cross connection is connected to a safety bursting disc.

According to an embodiment, this bursting disc is triggered when the pressure exceeds the safety pressure defined for the process, generally less than 150 bars (15 MPa).

According to an embodiment, a bursting disk tolerating 200 (20 MPa), 250 (25 MPa) or even 500 bars (50 MPa) of pressure can be fitted provided that all the elements of the reactor (seals, pumps, olive fittings . . . ) tolerate such pressure.

The reactive gas supply channel is equipped with a non-return valve, a quarter-turn valve, and a needle valve.

The reactive gas supply is ensured by a regulator capable of delivering the appropriate pressure. One of the nozzles of the shutter is connected to a substrate feed path (tubbing ⅛″ (0.3175 cm), ¼″ (0.635 cm), or 1″ (2.54 cm)).

This channel is equipped with a quarter-turn valve and a needle valve to precisely adjust the substrate feed rate (FIG. 3).

According to an embodiment (batch), this channel can remain closed.

According to another embodiment (continuous), this channel is connected to an HPLC type pump for flow rates lower than 1 ml/min to 300 ml/min which can deliver a pressure higher than the working pressure within the reactor with the liquid flow controlled automatically or to an industrial pump for flow rates from 300 ml/min to 50 L/min capable of delivering higher pressure to the working pressure within the reactor associated with a liquid flow meter in order to automatically control and regulate the liquid flow.

One of the nozzles of the shutter is connected to an inert gas supply path allowing the reactor to be purged (FIG. 3). This channel is fitted with a quarter-turn valve that can be automated.

According to a preferred embodiment, the reactions are carried out in an inert atmosphere and the inert gas is argon. According to an embodiment, this inert gas is nitrogen.

One of the shutter connections is connected to a degassing vent (FIG. 3). This degassing channel is fitted with an automated quarter-turn valve in order to depressurize the reactor.

The outlet of this channel must be positioned under a suction device in order to eliminate residual gases in complete safety.

One of the nozzles of the shutter is equipped with a tubing plunging into the reactor, allowing samples to be taken (FIG. 3). This diving tubing is equipped, at its submerged end, with a filter candle.

Advantageously, the filter candle consists of a threaded sintered hollow cylinder, the porosity of which can be between 2 and 50 μm, preferably between 5 and 50 μm. The sampling valve is a quarter-turn valve and a needle valve making it possible to recover a representative sample of the reaction mixture by overpressure.

This set is planned to be fully automated.

Advantageously, another of the nozzles of the shutter is used to introduce a immersion sleeve into the reactor which can be fitted with an indifferent probe. According to an embodiment, this probe can be a thermocouple.

The reactor has a threaded side outlet. According to a (continuous) embodiment, this orifice is fitted with a filter candle and connected to an outlet channel (tubbing ⅛″ (0.3175 cm) or ¼″ (0.635 cm) or even 1″(2.54 cm) to 2″ (5.08 cm)) to the downstream reactor of the cascade.

Stirring is ensured by a self-priming turbine of the Rushton type.

The stirring device consists of a hollow shaft and a hollow impeller.

This hollow turbine consists of two parallel stainless steel discs linked together by 5 to 7 vertical blades. According to an embodiment, these blades can be oriented parallel to the radius of the discs. According to another embodiment, these blades can be oriented by making an angle of 10 to 30 degrees with respect to the radius of the discs.

The rotation of the turbine, from a certain speed creates a depression downstream of the discs.

The pressurized hydrogen delivered into the dead volume of the reactor is then driven through the hollow shaft to the depression zone and distributed in the solvent in the form of small bubbles. Such a device, equipped with counter blades, ensures efficient gas-liquid transfer (FIG. 5).

The length of the stirring shaft is from 80 mm to 800 mm. The length of the stirrer depends on the volume of the reactor so for a volume of 100 ml the length is for example of the order of 80 mm and for a 200L reactor the length of the stirrer is for example about 80 cm.

This length is in particular from 80 mm to 200 mm, or from 80 mm to 600 mm, or from 200 mm to 600 mm or from 200 mm to 800 mm or from 600 mm to 800 mm.

The diameter of the discs ranges from 20 mm to 50 cm, preferably ranges from 20 mm to 40 cm and is adapted to the volume of the reactor.

A subject of the present invention is the use of the devices according to the invention, characterized in that the catalyst load can be different in each of the reactors. As also indicated, the catalyst load in a hydrogenation reaction can be different in each of the reactors, it can for example be in a ratio of 1;1.3;1.5 or even 0.7;1.7;2 in a cascade of three reactors. The catalyst load can be of the order of 1 mole % or a multiple of this percentage, for example in palladium.

The stirring speed can be of the order of 1,000 RPM (rotations per minute).

A speed of around 800 RPM can also be used.

Advantageously, the reactor can be provided with a temperature control device in order to work at the desired temperature.

According to an embodiment, this temperature is between −30° C. and 300° C. According to another embodiment, this temperature may be higher provided that the seals tolerate it.

According to an embodiment, this heating device can be a removable double jacket screwed to the reactor via two threaded holes (non-opening) drilled in the reactor.

This operating mode is preferred for low temperatures (−30° C. to 120° C.).

According to this same embodiment, the control of the temperature of the double jacket is ensured by a thermostatically controlled heat transfer fluid.

In the case of industrial reactors, the jacket is non-removable and is made of stainless steel.

According to another embodiment, this heating device can be a ceramic heating collar with anti-scalding plate connected to a control box.

This operating mode is preferred for high temperatures (120° C. to 300° C.).

The present invention therefore relates to the use of the devices according to the invention characterized in that the reaction temperature can be between −10 and 300° C., preferably a high temperature of at least 130° C. preferably by use either a double jacket or a heating collar.

The subject of the present invention is the use of the devices according to the invention, characterized in that the reaction temperature can be different in each of the reactors.

The reaction temperature, and in particular the temperature of the hydrogenation reaction, can in particular be of the order of 100° C., it can also be of the order of 80° to 120° C. As indicated, it can vary from one reactor to another in the cascade of reactors, for example it can be of the order of 80° C. for the first and the second reactor and of the order of 100° C. for the third reactor in a three-reactor system.

The temperature can also be of the order of 100° C. for the first, of the order of 110° C. for the second reactor and of the order of 130° C. for the third reactor in a system with three reactors.

According to embodiment, such a reactor can be used alone in batch mode provided that the feed path and the outlet orifice are blocked by elements that tolerate the working pressure of reactive gas (FIG. 3).

According to another embodiment, 1 to N (N natural integer) reactors of the same type can be connected in cascade in order to work in continuous flow (FIG. 2).

According to a preferred embodiment, the optimal number N of reactors to be connected to the cascade can be determined by a material balance on a single continuous reactor coupled with a kinetic study carried out in batch mode.

According to another embodiment, this number N can be determined empirically.

The present invention also relates to the use of the devices according to the invention, characterized in that the reaction is implemented so that the reactive gas pressure is between 10 bars (1 MPa) and 500 bars (50 MPa) preferably between 10 bars (1 MPa) and 250 bars (25 MPa) and more preferably between 10 bars (1 MPa) and 50 bars (5 MPa).

In hydrogenation reactions, the hydrogen pressure is preferably of the order of 20 bars (2 MPa) or 30 bars (3 MPa), values of 10 (1 MPa), 12 (1.2 MPa), 20 (2 MPa) and 50 (5 MPa) bar can also be used.

When three reactors are used, the respective pressures in each of the reactors can be of the order of 15 (1.5 MPa) in the first reactor, 12 (1.2 MPa) in the second reactor and 10 bars (1 MPa) in the third reactor, 20 (2 MPa) in the first reactor, 12 (1.2 MPa) in the second reactor, and 5 bar (0.5 MPa) in the third reactor or even 30 (3 MPa) in the first reactor, 28 (2.8 MPa) in the second reactor and 5 bar (0.5 MPa) in the third reactor.

The circulation of the reaction mixture through the N reactors of the cascade can be ensured by applying a decreasing pressure in each reactor.

Advantageously, the N reactors of the cascade can be fixed on their respective frames at decreasing heights to improve the circulation of the feed mixture.

The supply of the device with reaction mixture can be ensured by an HPLC pump or conventional industrial pump capable of delivering a pressure greater than the working pressure in reactive gas.

The adjustment of the feed and withdrawal flow rates can be refined by adjusting the opening of the needle valves positioned between each reactor.

At each connection between the reactors, there are valves that can be automated, and flow controls will be inserted between each reactor in order to control the flow between each reactor. The absence of circulation of the solid phase (catalyst) through the cascade is ensured by the presence of filter candles at each outlet orifice of the reactor.

In the event of clogging of the circulation path of the reaction mixture, a slight counter pressure can be exerted on the cascade to declog the intermediate filter candles.

According to an embodiment, this counterpressure can be implemented by temporarily applying an increasing pressure of reactive gas within the cascade.

Advantageously, a RAMAN or NIR type analytical probe can be integrated at the level of the connection between two reactors of the cascade in order to visualize in real time the efficiency of the process under pressure, and, if necessary, to plan a maintenance operation. (change of deactivated catalyst) (FIG. 2).

Advantageously, such a maintenance operation can be carried out without stopping the overall process but simply by disconnecting one of the reactors from the cascade in order to isolate it. Advantageously, an identical N+1th (N natural integer) reactor can be provided at the end of the cascade, the latter being put into service only during the maintenance operation of an upstream reactor in order to maintain a process with N reactors (N natural integer) in cascade and not to lose in level of performance.

All of the individual parameters (temperature, pressure, stirring speed, possibly catalytic load) of the N reactors (N natural integer) of the cascade can be both controlled and visualized in a completely independent manner.

According to an embodiment, it is possible to apply the same combination of operating parameters to the N reactors (N natural integer) of the cascade.

According to another embodiment, different parameters can be applied.

All of the fluid connections and gas connections are ensured by Swagelock type connector elements compatible with the reactive gas working pressure (olive fitting, union+washer and ferrules). According to one embodiment, all of these connections consist of ⅛″ (0.3175 cm), ¼″ (0.635 cm), or 1″ (2.54 cm) tubings. According to another embodiment, all of these connections consist of ⅛″ (0.3175 cm) tubing or even larger dimensions adapted to the volumes of the reactors.

The subject of the present invention is the use of the devices described above for carrying out reactions under pressure of the liquid-solid-gas and solid-liquid type, in particular hydrogenation, oxidation, carbonylation or amination reactions, preferably hydrogenation reactions.

The hydrogenation reactions are carried out in the presence of a catalyst such as the metals of the platinum group, in particular platinum, palladium, rhodium and ruthenium, for example the Wilkinson catalyst, based on rhodium or the catalyst of Crabtree based on iridium or Lindlar's catalyst based on palladium on calcium carbonate.

One can also use nickel-based catalysts such as Raney nickel or Urushibara nickel. Preferably, Raney Nickel, platinum on carbon (Pt/C) or a catalyst of the SiliaCat® type, in particular Siliacat Pd(0), is used.

Siliacat Pd(0) is a catalyst consisting of Pd trapped in a sol-gel system.

Specifically, highly dispersed Pd nanoparticles (uniformly in the range of 4.0-6.0 nm) are encapsulated in an organosilica matrix.

The structure of this catalyst is shown below.

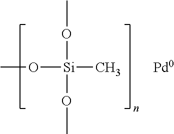

This catalyst is marketed by several companies including Dichrom GmbH in Germany and the Silicycle Company in Canada.

A particular subject of the present invention is the use of the devices of the invention for the continuous hydrogenation of adiponitrile to hexamethylene diamine in the presence of Raney nickel, characterized in that the process is implemented by using at least three reactors of different volumes with decreasing volumes and increasing masses of catalysts and temperatures depending on the reactors.

During the implementation of this reaction, the cascade of reactors preferably comprises three elements and the volume of the reactors is decreasing and is such that if the first reactor has a volume R1, the second reactor has a volume R2 equal to half of R1 and the third reactor has a volume R3 equal to one third of R1.

A subject of the present invention is in particular the use of the devices of the invention for the continuous hydrogenation of p-nitrophenol to p-aminophenol in the presence of a platinum-on-carbon (Pt/C) catalyst, characterized in that the process is implemented by using a cascade of two to five reactors, preferably with decreasing hydrogen pressure according to the reactors.

A subject of the present invention is in particular the use of the devices of the invention for the continuous acetylation of anisole to acetanisole using acetic anhydride in the presence of beta zeolite, characterized in that the method is implemented by using a cascade of at least two reactors and at a temperature of at least 130° C.

It is understood that the figures indicated above for information purposes (around) must be interpreted with a margin of appreciation of 10, 20 or even 25%.

The following examples are provided by way of illustration of the present invention. It is understood that the reference examples can be used to implement the invention as claimed, for example when a single reactor is used, a cascade of reactors of the same type but of different dimensions can be arranged in a cascade according to the invention.

### EXAMPLES

### 1) Reference Example: Hydrogenation of p-Nitrophenol to p-Aminophenol in Batch Mode

The reaction in batch mode is carried out in a single closed reactor.

The reactor is preloaded with a solution of 6.95 g of /nitrophenol in 100 mL of EtOH and 9.75 mg of Pt/C (Sigma Aldrich). The reactor is then purged of nitrogen (3 purges, 5-7 bar) then pressurized with hydrogen (H₂, Alphagaz, Air Liquide) under 15 bar.

Stirring is set at 1000 RPM and the reactor is heated to 80° C. by its double jacket for 1 h20.

At the end of the reaction, the reactor is inerted by purging the nitrogen and the reaction medium is analyzed by HPLC (reverse phase, column C 18).

Analysis shows a 92% conversion of p-nitrophenol to p-aminophenol with no trace of reaction co-product.

### 2) Continuous Hydrogenation of p-Nitrophenol to p-Aminophenol in a Cascade of Two to Five Perfectly Stirred Continuous Reactors

The same device is reused to carry out the reaction on a cascade of two perfectly stirred continuous reactors. The outlet channel of the first reactor, still equipped with a 5 μm filter candle in order to keep the catalytic load of the autoclave constant, is connected to the inlet of a second reactor similar to the first in all respects. The two reactors are loaded with 20 mg of Pt/C 10% w/w (Sigma Aldrich). A conversion of 50% is simulated in the first reactor (2.72 g of p-aminophenol for 3.48 g of p-nitrophenol) and a conversion of 75% is simulated in the second reactor (4 g of p-aminophenol for 1.8 g of p-nitrophenol).

Under the conditions previously described, but with a slightly decreasing pressure (80° C., 15 bars, 1000 RPM in the first reactor; 80° C., 12 bars, 1000 RPM in the second reactor), the cascade is fed with a solution of p-nitrophenol in ethanol (0.3 M) at a flow rate of 3 mL/min (residence time, 30 minutes per reactor) for 5 hours.

The withdrawal valve of the second reactor is adjusted so as to have an outlet flow rate approximately equal to the inlet flow rate. No event occurs during the 5 hours of reaction.

Samples are taken every 4 minutes. The HPLC analyzes show that the conversion oscillates between 70 and 83% for 20 minutes before stabilizing around 80% without formation of co-product. In another case, a third reactor is connected to the cascade.

Similarly, this reactor is loaded with 20 mg of Pt/C, and a starting conversion of 90% is simulated (4.9 g of p-aminophenol for 695 mg of p-nitrophenol).

In the conditions described above (80° C., 1000 RPM, 15 bar; 12 bar; 10 bar), the cascade is fed for 4 hours at a flow rate of 4 mL/min (residence time 25 minutes). No event occurs. At the reactor outlet, samples are taken every 4 minutes. The HPLC analyzes show that the conversion oscillates between 80 and 96% for 20 minutes before stabilizing at 95% for 4 hours.

### 3) Reference Example: Hydrogenation of p-Nitrophenol to p-Aminophenol in Batch Mode with a Siliacat Pd(0) Catalyst

The reaction in batch mode is carried out on a single closed reactor.

The reactor is preloaded with a solution of 6.95 g of p-nitrophenol in 100 mL of EtOH (Aldrich) and 0.208 mg of SiliaCat Pd(0) (Silicycle). The reactor is then purged of nitrogen (3 purges, 5-7 bar) then pressurized with hydrogen (H₂ Alphagaz, Air Liquide) under 15 bar. Stirring is set at 1000 RPM. When the reactor is heated to a temperature of 80° C., a conversion of 86% is obtained in 80 min and when the reaction is carried out at 100° C., a conversion of 88% is obtained in 60 min.

### 4) Hydrogenation of p-Nitrophenol to p-Aminophenol in Continuous Mode with a SiliaCat Pd(0) Catalyst on 2 Continuous Reactors

Following the kinetic study, a simulation of a third reactor was carried out taking into account the criteria of the first 2 reactors.

The results obtained are shown in the table below

| | | | | | Operating | |
|---|---|---|---|---|---|---|
| | Operating | | Operating | | conditions R3 | |
| Parameters | conditions R1 | | conditions R2 | | (modeling) | |
| Volume | 0.1 | L | 0.15 | L | 0.4 | L |
| Flow rate | 12 | ml/mn | 12 | ml/mn | 12 | ml/mn |
| Pressure | 20 | bar | 32 | Bar | 5 | Bar |
| Temperature | 100 | ° C. | 110 | ° C. | 130 | ° C. |
| Mcata Siliacat Pd | 0.70% | | 1.70% | | 2% | |
| Con p- | 3 | mol/l | 0.64 | mol/l | 0.163 | mol/l |
| Nitrophenol | | | | | | |
| Conc p-Nitro | 0.64 | mol/l | 0.16 | mol/l | 0.011 | mol/l |
| Conversion | 0.36 | | 0.73 | | 0.93 | |
| Overall | 35.95 | | 83.62 | | 98.85 | |
| conversion (%) | | | | | | |

In the table above, the quantity of “Mcata” catalyst is expressed in mol %.

A productivity of 3.7 kg/L/day of p-aminophenol is obtained with 3 reactors in series.

FIG. 8B shows the conversions for each reactor

### 5) Hydrogenation of Adiponitrile in Hexamethylene Diamine Continuously with NiRa with Holistic Optimization of the Operating Conditions with Design of the Reactor

One operates with 3 different reactors. The volume of each reactor is 2 liters, 1.5 liters and 1 liter respectively. The reaction temperature is 80° C. for the first 2 reactors and 100° C. for the third reactor and the mass of catalyst is 10 g, 13 g and 15 g respectively. The flow rate is 0.11/s. The productivity is 9.29 kg HMD/Liter/hour.

### 6) Hydrogenation of Pure p-Nitrosophenol and a Mixture of Para and Ortho-Nitrosophenol

A mixture of para and ortho-nitrosophenol (ratio o/p=10/90) was dissolved in MeOH and Pt/(C) (% mass) was suspended.

The mixture was then placed under hydrogen (1 atm) with stirring. After 2 hours, the mixture no longer showed traces of 2-nitrosophenol and 4-nitrosophenol. The solution was filtered through Celite, and evaporated to dryness to obtain a mixture of 2-aminophenol and 4-aminophenol in a ratio of o/p=10/90. Two other tests of hydrogenation of pure p-nitrosophenol were carried out under pressure (P=15bar) at T=80° C., using the Pt/C catalyst and the SiliaCat Pd(0) catalyst.

A conversion of the order of 99.9% was obtained with an excellent yield of 99.8% (control by HPLC).

### 7.1) Carboxylation of Propylene Oxide with a Solid Catalyst to Form Propylene Carbonate in Batch Mode

The catalyst used is DEAE 1ER from Merck Sigma Aldrich with a catalytic load of 76 g/L. In a 100 mL reactor, the pressure is 75.4 bar and the temperature 95° C. The reaction is carried out without solvent.

The selectivity is greater than 99% and the reaction time is 60 hours.

### 7.2) Carboxylation of Propylene Oxide with a Solid Catalyst to Form Propylene Carbonate in a Continuous Mode

The catalyst used is diethylaminoethyl cellulose.

The results obtained are shown in the table below

| | | PO | PO | Conversion | Overall | Cat | | | Residence |
|---|---|---|---|---|---|---|---|---|---|
| | Volume | Conce | Concs | rate | conversion | mass | | | time |
| | (Liter) | (mol/l) | mol/l | (%) | rate (%) | (g) | T (° C.) | P Bar | (hours) |
| Reactor Nº1 | 1 | 12 | 4 | 66.6666667 | 66.6666667 | 110 | 95 | 8.5 | 5 |
| Reactor Nº2 | 1.2 | 4 | 1.5 | 62.5 | 87.5 | 130 | 95 | 8 | 6 |
| Reactor Nº3 | 1.4 | 1.5 | 0.5 | 66.6666667 | 95.8333333 | 140 | 95 | 7.5 | 7 |
| Reactor Nº4 | 1.6 | 0.5 | 0.1 | 80.00 | 99.17 | 160 | 95 | 7 | 8 |
| | 5.2 | | | | | 540 | | | 25 |

A productivity of 0.0174 kg of propylene carbonate/L/H/kg of catalyst is obtained.

### 8.1) Ammonolysis of ethyl 2-(2-pyrrolidone)-butyrate (PBE) to 2-(2-oxopyrolidin-1-yl)butyramide (Etiracetam) in Batch Mode

The reaction is catalyzed by sodium methanolate MeONa. Ethyl 2-(2-pyrrolidone)-butyrate (1 equivalent) is dissolved in methanol (0.3 volume). Sodium methoxide (0.04 equivalent) and ammonia (3.3 equivalents) are then introduced together. The recycled ammonia is loaded and supplemented with the necessary quantity of fresh ammonia. The reactor is then heated by the double jacket so as to maintain a pressure below 6 bars. (Heating to 60° C. in 1 h-1 h30).

The reaction medium is cooled to 0° C. (acceptable temperature range: −11° C. to 10° C.) and, at the same time, the ammonia is degassed and recycled. Ammonia is condensed in an evaporator containing methanol. The ammonia solution in methanol will be used during the following synthesis. The medium is then filtered and washed with methanol.

The final product is dried under reduced pressure or at atmospheric pressure (the final internal temperature is 60±20° C.).

The optimal operating conditions are indicated in the table below

| Exp | | | | | |
|---|---|---|---|---|---|
| condition | MeOH | NH₃ | PBE | MeoNa | Total |
| V(ml) | 219.287 | 123.943 | 673.035 | 56.960 | 1073.22488 |
| Mass (g) | 174.545 | 98.591 | 708.635 | 28.480 | 1010.25127 |
| mol | 5.447 | 5.803 | 3.560 | 0.498 | 15.308 |
| Eq | 1.530 | 1.630 | 1.000 | 0.140 | NA |
| [ ] (mol/g) | 0.005 | 0.006 | 0.004 | 0.000 | NA |
| [ ] (g/l) | 206.029 | 116.069 | 631.379 | 46.523 | NA |
| [ ] (g/g) | 0.174 | 0.098 | 0.704 | 0.025 | NA |

### 8.2) Ammonolysis of ethyl 2-(2-pyrrolidone)-butyrate (PBE) to 2-(2-oxopyrolidin-1-yl)butyramide (Etiracetam or ETI) in Continuous Mode

The reaction is catalyzed by sodium methanolate MeONa. The optimal operating conditions are indicated in the table below:

| | | Inlet | Outlet | | | | |
|---|---|---|---|---|---|---|---|
| | | PBE | PBE | | | | Total |
| | Volume | Conce | Concs | | | | numb |
| | (Liter) | (mol/l) | mol/l | PBM | ETI | Etoh | moles |
| Reactor Nº1 | 1 | 3.5 | 0.35 | 1.8 | 0.3 | 2.95 | 3.05 |
| Reactor Nº2 | 1.3 | 0.35 | 0.1 | 0.55 | 2.8 | 3.45 | 3.45 |
| Reactor Nº3 | 1.5 | 0.1 | 0 | 0 | 3.5 | 3.45 | 3.5 |
| | 3.8 | | | | | | |

| | Conversion | Overall | Cat | | | Residence |
|---|---|---|---|---|---|---|
| | rate | conversion | mass | | | time |
| | (%) | rate (%) | (g) | T (° C.) | P Bar | (Minutes) |
| Reactor Nº1 | 50 | 78.7878788 | 10 | 117 | 8.5 | 15 |
| Reactor Nº2 | 71.4285714 | 93.9393939 | 13 | 120 | 8 | 19.5 |
| Reactor Nº3 | 100 | 100 | 15 | 125 | 7.5 | 22.5 |
| | | | 38 | | | 57 |

The productivities obtained are indicated in the table below:

| | | |
|---|---|---|
| Flow L/H | 4 | |
| Residence time M | 57 | |
| production | 14 | mol/h |
| PRODUCTION | 2382.8 | g/h |
| | 627.052632 | G/H/L |

### 9.1) Oxidation of Benzyl Alcohol to Benzaldehyde in Batch Mode

The operating conditions are as follows: 485 mmol of benzyl alcohol (50 mL) are introduced into a 50 mL reactor, at a temperature of 85° C., at 800 RPM with a O₂ pressure of 4 bars (400,000 Pa) and with a catalytic load of 0.125% Pd catalyst (SiliaCat Pd(0)) for 1 h.

The conversion is 100% and the selectivity is 83%.

### 9.2) Oxidation of Benzyl Alcohol to Benzaldehyde in Continuous Mode

The operating conditions in the first reactor (reactor volume of 100 mL) are: benzyl alcohol (964 mmol), 0.25 mol % of Pd (Silicat Pd (0)), T=85° C., P=4 bars (400,000 Pa) of O₂, 1000 RPM with a flow rate of 3 mL/min.

In this first reactor, a conversion of 70% and a selectivity of 55% are obtained.

The conditions in the second reactor (reactor volume of 75 mL) are as follows: benzyl alcohol (289 mmol), 0.188 mol % of Pd (Silicate Pd (0)), T=85° C., P=10 bar (1 MPa) of O₂, 1000 RPM with a flow rate of 3 mL/min.

A conversion of 94% and a selectivity of 84% are obtained in this second reactor.

The conditions in the third reactor (50 mL reactor volume) are as follows: benzyl alcohol (58 mmol), 0.188 mol % Pd (Silicate Pd (0)), T=85° C., P=10 bar (1 MPa) of O₂, 1000 RPM with a flow rate of 3 mL/min.

In this third reactor, a conversion of 100% and a selectivity of 88%.

The volumetric productivity of benzaldehyde obtained is 23.98 kg/l/h, with a yield of 88%.

### 10.1) Suzuki-Miyaura Reaction with Phenylboronic Acid in Batch Mode

3 Different iodoaryls were tested: 2-iodothiophene, 2-iodobenzene and 4-iodobenzoic acid.

In a 100 mL reactor, an iodoaryl (3.0 mmol), phenylboronic acid (6.0 mmol), the Pd—Cu/C catalyst (43.0 mg, ca. 4.0 mmol), and K₃PO₄ (12.0 mmol) are added in 50 mL of ethanol.

The reaction medium is heated at 78° C. for 3 h under an inert atmosphere (nitrogen).

At the end of the reaction, the reaction medium is filtered. Then the solvent is evaporated.

For 2-phenylthiophene, the yield is 96.7% with a yield relative to Pd/C of 78.2%.

For biphenyl, the yield is 97.5% with a yield relative to Pd/C of 96.7%.

For 1,4-biphenylcarboxylic acid, the yield is 88.7% and the yield with respect to Pd/C is 85.1%.

### 10.2) Suzuki-Miyaura Reaction with Phenylboronic Acid in Continuous Mode

3 different iodoaryls were tested: 2-iodothiophene, 2-iodobenzene and 4-iodobenzoic acid.

The operating conditions in reactor No. 1 are T=120° C., P=5 bars (500,000 Pa), mass of Pd—Cu/C catalyst=50 mg with a reactor volume of 100 ml, to obtain a conversion of 50% with a volume of 100 ml. The operating conditions of reactor No. 2 are T=110° C., P=3.5 bar (350,000 Pa), mass of catalyst Pd—Cu/C=75 mg with a reactor volume of 150 ml, to obtain a 90% overall conversion. And finally the operating conditions of reactor No. 3 are T=105° C., P=2 bars (200,000 Pa), mass of catalyst Pd—Cu/C =100 mg with a reactor volume of 200 ml, to obtain a conversion overall 100%.

The input flow is composed of the compound iodoaryl (3.0 mmol), phenylboronic acid (6.0 mmol), and K₃PO₄ (12.0 mmol) in 50 mL EtOH with a flow rate of 8 ml/min.

### 11.1) Heck Reaction with 2-iodothiophene in Batch Mode

Three different compounds were tested: styrene, phenyl acetylene and methylbutynol.

The catalyst used for styrene and methylbutynol is Pd—Cu/C. The catalyst used for phenyl acetylene is Pd—Ag/C. In a 100 mL reactor, 2-iodothiophene is introduced (3.0 mmol), the alkene or alkyne (6.0 mmol), the catalyst (43.0 mg, ca. 4.0 mmol), and triethylamine (6.0 mmol) are added in 50 mL of acetonitrile. The reaction medium is heated at 82° C. for 3 hours under a nitrogen atmosphere. At the end of the reaction, the reaction medium is filtered.

Then the solvent is evaporated. For 2-styrylthiophene, the yield is 88.8%. For acetylphenylthiophene, the yield is 88.8%. For 2-thiophene methylbutynol, the yield is 94.2%

### 11.2) Heck Reaction with 2-iodothiophene in Continuous Mode

Three different compounds were tested: styrene, phenyl acetylene and methylbutynol.

The catalyst used for styrene and methylbutynol is Pd—Cu/C. The catalyst used for phenyl acetylene is Pd—Ag/C. The operating conditions in reactor No. 1 are T=120° C., P=5 bars (500,000 Pa), catalyst mass of 50 mg with a reactor volume of 100 ml, to obtain a conversion of 50% with a volume of 100 ml. The operating conditions of reactor No. 2 are T=110° C., P>5 bars (500,000 Pa), catalyst mass of 75 mg with a reactor volume of 150 ml, to obtain an overall conversion of 90%. And the operating conditions of reactor No. 3 are T=105° C., P>4 bars (400,000 Pa), catalyst mass of 100 mg with a reactor volume of 200 ml, to obtain an overall conversion of 100%. The input flow is composed of 2-iodothiophene (3.0 mmol), alkene or alkyne (6.0 mmol), and triethylamine (6.0 mmol) are added in 50 mL of acetonitrile with a flow rate of 8 ml/min.

1. A Device for chemical reactions under pressure or high pressure and/or under high temperature in continuous flow comprising a cascade of N autoclave reactors interconnected, wherein the N reactors of the cascade are provided with means allowing them to be individually controlled in a completely independent manner, it being understood that N is a natural integer greater than 1 and that the cascade of reactors comprises at least two reactors of different volumes, increasing or decreasing in the direction of the flow of the fluids, the said chemical reactions being of the Gas-Liquid-Solid type or of the Liquid-Solid type, said device comprising between each of said reactors means allowing the fluid phase to be in continuous flow and allowing the solid phase to be in batch, said means being a system of filter candles.

2. The Device according to claim 1, wherein each reactor is provided with a liquid inlet and outlet, and with a possible reactive gas inlet, a bursting disc, a vent, an immersion sleeve for parameter measurement, a sampling valve, a double jacket, a heating collar and a valve placed at the bottom of each reactor and making it possible to withdraw the deactivated catalyst and to replace it with a new catalyst, each reactor comprising at its outlet and inside itself, a filter, in particular a frit.

3. The Device according to claim 1, wherein the liquid outlet orifice is provided with a system of filter candles, with a porosity of between 2 and 50 μm

4. The Device according to claim 1, wherein an online analysis tool PAT (Process Analytical Technology) by UV, NIR, Raman or any other analysis technique is positioned between each reactor.

5. The Device according to claim 1, wherein an N+1th reactor can be positioned at the end of the cascade and connected to the process during maintenance operations requiring one of the reactors of the cascade to be isolated.

6. The Device according to claim 1, for carrying out reactions under high pressure, wherein in the cascade of reactors, the volume of the reactors is decreasing and is such that when N is equal to or greater than 3, if the first reactor has a volume R1, the second reactor has a volume R2 comprised between R1 and 0.5 R1 and the third reactor has a volume R3 comprised between 0.8 R1 and 0.4 R1.

7. A Method for using a device for chemical reactions under pressure or high pressure and/or under high temperature in continuous flow comprising a cascade of N autoclave reactors interconnected, wherein the N reactors of the cascade are provided with means allowing them to be individually controlled in a completely independent manner, it being understood that N is a natural integer greater than 1 and that the cascade of reactors comprises at least two reactors of different volumes, increasing or decreasing in the direction of the flow of the fluids, the said chemical reactions being of the Gas-Liquid-Solid type or of the Liquid-Solid type, said device comprising between each of said reactors means allowing the fluid phase to be in continuous flow and allowing the solid phase to be in batch comprising, for carrying out reactions in which the heat of reaction is greater than 50 kJ/mol, characterized in that wherein in the cascade of reactors, the volume of the reactors is increasing and is such that when N is equal to or greater than 3, if the first reactor has a volume R1, the second reactor has a volume R2 comprised between 1.25 R1 and 1.5 R1 and the third reactor has a volume R3 comprised between 1.5 R1 and 4 R1.

8. The Method for using a device according to claim 7 comprising, carrying out reactions under pressure or high pressure of the liquid-solid-gas and solid-liquid type, in particular hydrogenation, oxidation, carbonylation, carboxylation, amination, in particular ammonolysis, Heck or Suzuki-Miyaura, preferably hydrogenation reactions.

9. The Method for using a device according to claim 7 comprising, carrying out Gas-Liquid-Solid reactions, in which each reactor is provided with stirring by a hollow self-priming turbine ensuring dispersion of the reactive gas in the reaction medium thanks to a depression created by the blades of the agitator and in that the stirring speed is sufficient to overcome the pressure drop and is preferably greater than 300 rpm, in particular 500 rpm.

10. The Method for using a device according to claim 7 comprising, carrying out reactions under pressure or high pressure of the liquid-solid-gas type, in particular hydrogenation, oxidation, carbonylation, carboxylation or amination, in particular ammonolysis, preferably hydrogenation reactions.

11. The Method for using a device according to claim 7 comprising, carrying out reactions at high temperature of the solid liquid type, in particular Heck and Suzuki-Miyaura reactions.

12. The Method for using a device according to claim 7, wherein the reaction is implemented so that the reactive gas pressure is between 2 bars (0.2 MPa) and 500 bars (50 MPa) preferentially between 2 bars (0.2 MPa) and 250 bars (25 MPa) and more preferentially between 2 (0.2 MPa) and 50 bars (5 MPa).

13. The Method for using a device according to claim 7, wherein the reaction temperature is between −10 and 300° C., preferably a high temperature of at least 130° C., preferably by use either of a double jacket, or a heating collar, and in that the reaction temperature and the catalyst load can be different in each of the reactors.

14. The Method for using a device according to claim 7, wherein the reaction is implemented so the reactive gas pressure is between 1 bar (0.1 MPa) and 100 bars (10 MPa) preferably between 1 bar (0.1 MPa) and 50 bars (5 MPa) and more preferably between 1 bar (0.1 MPa) and 30 bars (3 MPa).

15. The Method for using a device according to claim 7, wherein the reaction temperature is between −10 and 300° C., preferably a high temperature of at least 130° C., preferably by use either of a double jacket, or a heating collar, and in that the reaction temperature and the catalyst load can be different in each of the reactors.

16. The Method for using a device according to claim 7 for the continuous hydrogenation reaction of adiponitrile to hexamethylene diamine in the presence of Raney nickel, wherein the method is implemented by using at least three reactors of different volumes with decreasing volumes and increasing masses of catalysts and temperatures depending on the reactors.

17. The Method for using a device according to claim 7, wherein the cascade of reactors comprises three elements and in that the volume of the reactors is decreasing and is such that if the first reactor has a volume R1, the second reactor has a volume R2 equal to half of R1 and the third reactor has a volume R3 equal to a third of R1.

18. The Method for using a device according to claim 7 for the continuous hydrogenation reaction of p-nitrophenol to p-aminophenol in the presence of a platinum-on-carbon (Pt/C) catalyst, wherein the method is implemented by using a cascade of two to five reactors, preferably with decreasing hydrogen pressure depending on the reactors.

19. The Method for using a device according to claim 7 for the continuous acetylation reaction of anisole to acetanisole using acetic anhydride in the presence of beta zeolite, wherein the method is implemented by using a cascade of at least two reactors and at a temperature of at least 130° C. or for the continuous ammonolysis reaction of ethyl 2-(2-pyrrolidone)-butyrate to 2-(2-oxopyrolidin-1-yl)butyramide in the presence of sodium methanolate wherein the method is implemented by using a cascade of at least two reactors at a pressure of at least 7.5 bars (0.75 MPa) and at a temperature of at least 117° C., or for the continuous oxidation reaction of benzyl alcohol to benzaldehyde using a SiliaCat Pd(0) palladium catalyst, wherein the method is implemented by using a cascade of at least 2 reactors and at a pressure of at least 10 bars (1 MPa), in particular at a temperature of 85° C., or for the carboxylation reaction of propylene oxide to propylene carbonate using a diethylaminoethyl cellulose catalyst, wherein the method is implemented by using a cascade of at least two reactors and at a pressure of at least 7 bars (0.7 MPa) and at a temperature of at least 95° C. or for the continuous Suzuki-Miyaura reaction of a boronic acid with an iodoaryl using a Pd—Cu/C catalyst, wherein the method is implemented by using a cascade of at least two reactors and at a temperature of at least 105° C., in particular at a pressure of 2 bars (0.2 MPa) or for the continuous Heck reaction of an alkenyl or an alkyne with an iodoaryl using a palladium catalyst Pd-M/C with M a metal, wherein the method is implemented by using a cascade of at least two reactors and at a temperature of at least 105° C., in particular at a pressure of 4 bars (0.4 MPa).

20-24. (canceled)
